(19) United States
(12) Patent Application Publication
(10) Pub. No.: US 2024/0315566 A1
(43) Pub. Date: Sep. 26, 2024

(54) OPTICAL SYSTEM FOR BREAST X-RAY COMPUTED TOMOGRAPHY

(71) Applicants: Izotropic Corporation, Surrey (CA); The Regents of the University of California, Oakland, CA (US)

(72) Inventors: Younes Achkire, Surrey (CA); John M. Boone, Oakland, CA (US); Andrew M. Hernandez, Oakland, CA (US)

(73) Assignees: Izotropic Corporation, Surrey (CA); The Regents of the University of California, Oakland, CA (US)

(21) Appl. No.: 18/123,462

(22) Filed: Mar. 20, 2023

(51) Int. Cl. A61B 5/00; A61B 6/04

(52) U.S. Cl. CPC A61B 5/0091; A61B 5/4312; A61B 6/0435

(57) ABSTRACT

An x-ray system includes a base component, a table to support a patient in a prone position and defining an opening for a breast of said patient to extend downwards therethrough into an active spatial region, a rotatable x-ray assembly including an x-ray source and an x-ray detector and being configured to rotate at least partially around said active spatial region, and an optical system that includes an optical detector arranged in an optical path to said opening of said table. In one aspect, the x-ray system further includes a radiation-safety interlock device configured to communicate with said optical detector and said x-ray source, allow said x-ray source to be engaged while an object is detected, and disengage said x-ray source otherwise. In another aspect, the optical system includes an optical detector that is optically conjugate with a radiation emission region of said x-ray source.

### BACKGROUND

### 1. Technical Field

Currently claimed embodiments of the invention relate to systems and components for breast examination and procedures, and more particularly to systems that have optical detectors.

### 2. Discussion of Related Art

While the current state-of-the-art for breast imaging is typically digital mammography, sometimes coupled with limited angle tomography which is often called breast tomosynthesis, it is recognized by the breast imaging community that these two-dimensional or pseudo-three-dimensional imaging modalities do not fully address the needs of breast cancer detection, diagnosis, and evaluation. Several groups have studied the use of computed tomography principles for breast imaging. These studies generally describe imaging a single breast at a time with the patient laying prone on a table, with the patient's breast hanging through a hole in the table in the so-called pendant position. An x-ray CT system then rotates around the pendant breast and acquires data which is then reconstructed into a three-dimensional image.

However, such conventional breast CT systems and both image quality and dosimetry are sensitive to patient position. There thus remains a need for improved breast CT systems.

### SUMMARY

An embodiment of the present invention is an x-ray system for at least one of breast examinations and procedures, that includes a base component and a table configured to support a patient in a prone position. The table is disposed proximate the base component with a space reserved therebetween and defines an opening that is positioned for a breast of the patient to extend downwards therethrough at least partially into an active spatial region. The x-ray system further includes a rotatable x-ray assembly disposed between the base component and the table, the rotatable x-ray assembly having an x-ray source and an x-ray detector and being configured to rotate the x-ray source and the x-ray detector at least partially around the active spatial region about an axis of rotation. The x-ray system further includes an optical system having an optical detector arranged in an optical path to the opening of the table, and a radiation-safety interlock device configured to communicate with the optical detector and the x-ray source. The optical system is configured to detect a presence of an object at least one of in or covering the opening defined by the table, and the radiation-safety interlock device allows the x-ray source to be engaged while the presence of the object is detected and disengages the x-ray source otherwise.

Another embodiment of the present invention is an x-ray system for at least one of breast examinations and procedures, that includes a base component and a table configured to support a patient in a prone position and disposed proximate the base component with a space reserved therebetween. The table defines an opening that is positioned for a breast of the patient to extend downwards therethrough at least partially into an active spatial region. The x-ray system further includes a rotatable x-ray assembly disposed between the base component and the table, the rotatable x-ray assembly having an x-ray source and an x-ray detector and being configured to rotate the x-ray source and the x-ray detector at least partially around the active spatial region about an axis of rotation. The x-ray system further includes an optical system having a first imaging optical detector arranged with an imaging plane that is optically conjugate with a radiation emission region of the x-ray source and a second imaging optical detector arranged in an optical path to the opening of the table. The optical system further includes an image processor configured to communicate with the first imaging optical detector and the second imaging optical detector to provide information of the breast of the patient when the breast is extended downwards through the opening defined by the table.

Another embodiment of the present invention is an optical system configured for an x-ray system, that includes a first imaging optical detector configured to be arranged with an imaging plane that is optically conjugate with a radiation emission region of an x-ray source of said x-ray system, and a second imaging optical detector configured to be arranged in an optical path to an opening defined by a table of said x-ray system, the table being configured to support a patient in a prone position, the opening being positioned for a breast of the patient to extend downwards therethrough at least partially into the radiation emission region. The optical system further includes an image processor configured to communicate with the first imaging optical detector and the second imaging optical detector to provide information of the breast of the patient when the breast is extended downwards through the opening defined by the table.

Another embodiment of the present invention is a method, that includes providing an x-ray system comprising an optical system, acquiring optical data of a breast of a patient using the optical system of the x-ray system while the breast extends downwards through an opening defined by a table of the x-ray system, the table being configured to support said patient. The method further includes determining information about the breast of the patient using the optical data, and controlling at least one parameter of the x-ray system during the at least one of breast examinations and procedures based on the determined information about the breast of the patient.

### BRIEF DESCRIPTION OF THE DRAWINGS

Further objectives and advantages will become apparent from a consideration of the description, drawings, and examples.

FIG. 1 shows an example of an x-ray system for at least one of breast examinations and procedures, according to some embodiments of the current invention.

FIG. 2 shows another example of an x-ray system for at least one of breast examinations and procedures, according to some embodiments of the current invention.

FIG. 3 shows another example of an x-ray system for breast examinations and procedures, according to some embodiments of the current invention.

FIG. 4 shows an example of an optical system, according to some embodiments of the current invention.

FIG. 5 shows another example of an x-ray system for breast examinations and procedures, according to some embodiments of the current invention.

FIG. 6 shows an example of an imaging optical detector, according to some embodiments of the current invention.

FIG. 7 shows another example of an x-ray system for breast examinations and procedures, according to some embodiments of the invention.

FIG. 8 illustrates a process for performing at least one of breast examinations and procedures, according to some embodiments of the current invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed, and other methods developed, without departing from the broad concepts of the current invention.

All references cited anywhere in this specification, including the Background and Detailed Description sections, are incorporated by reference as if each had been individually incorporated.

The term “active spatial region” of an x-ray system is intended to refer to a region of space where at least a portion of an object or a subject can be positioned during x-ray breast examinations and procedures. For example, the active spatial region may be defined by the field of view (FOV) of the x-ray source and x-ray detector. The active spatial region may also be referred to as the “imaging FOV.” The active spatial region may also be defined as a radiation emission region of an x-ray source, which is the region that is directly irradiated by the primary x-ray beam generated by the x-ray source.

The term “base component” is intended to be a structural support for a rotating assembly. For example, the base component may support a bearing for a rotating gantry. In some embodiments, the base component can be placed upon a floor, or is a floor, of a room in which the rotating assembly is located. The term “base support” may be used equivalently to the term “base component.”

In some embodiments, the “x-ray assembly” can include an x-ray source and/or an x-ray detector. In some embodiments, the x-ray assembly can also include a rotating gantry to which the x-ray source and the x-ray detector are mounted. For example, in some embodiments, the x-ray detector can be a flat-panel detector. In some embodiments, the x-ray assembly includes a shield that substantially encloses the x-ray source and the x-ray detector. The term “rotatable x-ray assembly” may include, but is not limited to, systems for computed tomography (CT), cone-beam CT (CBCT), fan-beam CT, radiation therapy (e.g., x-ray therapy), and x-ray surgery (e.g., biopsy).

The term “shield enclosure” is intended to refer to an enclosure that substantially encloses the x-ray source and x-ray detector of an x-ray assembly and provides radiation protection to persons in proximity to the x-ray assembly. In some embodiments, the radiation shield enclosures described in U.S. patent application Ser. No. 17/727,540 can be used. U.S. patent application Ser. No. 17/727,540, which was filed on Apr. 22, 2022, is incorporated herein by reference in its entirety.

The term “linear motor” is intended to refer to a type of electric motor that has its stator and rotor “unrolled,” in a sense. For example, instead of producing a torque (rotation), a linear motor produces a linear force along its length. The length of the linear motor can be arranged as a straight or as a curved line. In some embodiments, the length of the linear motor can be in a closed loop such as, but not limited to, a circular loop. Synchronous linear motors are a type of linear motors with a stationary magnetic rail that acts as the stator, and a moving electromagnetic coil that acts as the rotor. In some embodiments, the linear motor described in U.S. patent application Ser. No. 17/942,895 can be used. U.S. patent application Ser. No. 17/942,895, which was filed on Oct. 18, 2022, is incorporated herein by reference in its entirety.

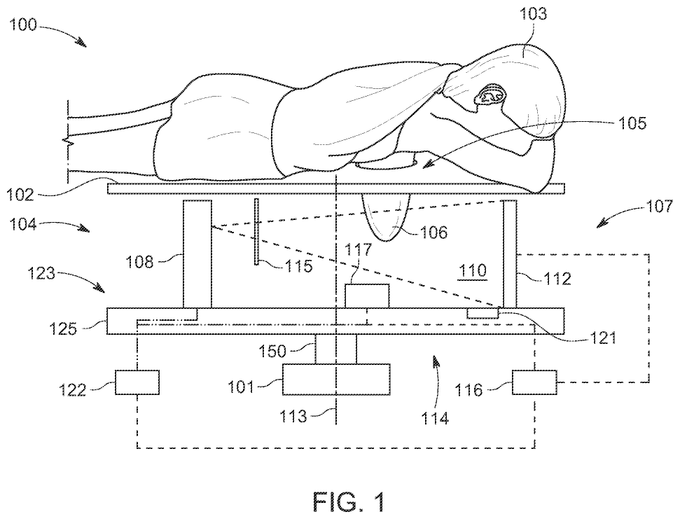

FIG. 1 shows an example of an x-ray system 100 for at least one of breast examinations and procedures, according to some embodiments of the current invention. The x-ray system 100 includes a base component 101 and a table 102 configured to support a patient 103 in a prone position, the table 102 being disposed proximate to the base component 101 with a space 104 reserved therebetween. The table 102 defines an opening 105 therein that is positioned for a breast 106 of the patient 103 to extend downwards therethrough at least partially into an active spatial region.

The x-ray system 100 also includes a rotatable x-ray assembly 107 disposed in the space reserved between the base component 101 and the table 102. In some embodiments, the base component 101 and the table 102 are configured to be fixed relative to each other, defining the space 104 therebetween to accommodate the rotatable x-ray assembly 107.

The rotatable x-ray assembly 107 includes an x-ray source 108 that generates an x-ray beam 110, and an x-ray detector 112 that is positioned to receive the x-ray beam 110. The x-ray assembly 107 is configured to rotate the x-ray source 108 and the x-ray detector 112 at least partially around the active spatial region about an axis of rotation 113.

In some embodiments, the x-ray system 100 may be configured to perform cone-beam computed tomography (CBCT). In some such embodiments, the x-ray source 108 may be a CT x-ray tube, the x-ray detector 112 may be a flat panel detector, and the x-ray beam 110 generated by the x-ray source 108 may be a cone beam.

In some embodiments, the axis of rotation 113 does not intersect the breast 106. In other embodiments, the position of the opening 105 and the table 102 may be configured so that the axis of rotation 113 intersects at least part of the breast 106 as the breast 106 extends downwards into the active spatial region.

The x-ray source 108 is positioned to irradiate, with the x-ray beam 110, at least a portion of the active spatial region into which the breast 106 extends. The x-ray detector 112 is positioned to receive at least a portion of the incident x-ray beam 110 after passing through the active spatial region and at least a portion of the breast 106. The x-ray beam 110 may be collimated by a collimator 115 before irradiating at least a portion of the breast 106 and thereafter impinging on the x-ray detector 112. The x-ray detector 112 may also be communicatively connected to a processor 116 which receives signals from the x-ray detector 112 and processes the signals received therefrom, in order to, for example, generate x-ray images of the breast 106.

The x-ray system 100 includes an optical system 114 having an optical detector 117 arranged in an optical path to the opening 105 of the table 102. The optical system 114 may also include the processor 116 in some embodiments. The optical path between the optical detector 117 and the opening 105 of the table 102 may be parallel to the axis of rotation 113. In the example shown in FIG. 1, the optical detector 117 is not aligned with the axis of rotation 113. However, in other embodiments, the optical detector 117 may be aligned with or substantially coincide with the axis of rotation 113. In some embodiments, the optical detector 117 is attached to the rotatable x-ray assembly 107 so as to rotate about the axis of rotation 113 in a substantially fixed relationship to the x-ray source 108 and the x-ray detector 112.

In some embodiments, the optical detector 117 is an imaging optical detector (e.g., an optical camera, an infrared camera, etc.) that is configured to provide an image of the breast 106, the opening 105, or both, within an imaging field of view of the optical detector 117. For example, the optical detector 117 may be communicatively connected to the processor 116 (as indicated in FIG. 1 by a dashed line therebetween), that receives imaging data from the optical detector 117, and processes the imaging data to generate optical images therefrom.

In some embodiments, the optical detector 117 is an optical sensor that is configured to provide a signal that varies based on how much light can be detected through the opening 105, depending on whether the opening 105 is empty, closed by a cover or shield, or at least partially occluded by the breast 106 of the patient 103 or by another object (e.g., a calibration phantom, etc.). The optical detector 117 may be communicatively connected to the processor 116 (as indicated in FIG. 1 by a dashed line therebetween), to receive and process the signal from the optical detector 117.

In some embodiments, the optical system 114 also includes an illumination system 121 arranged to illuminate the breast 106, the x-ray detector 112, or both. The illumination system 121 provides enough illumination for the optical detector 117 to optically detect the breast, even when the x-ray system 100 is sealed and a patient 103 is on the table 102.

The optical system 114 may also include a radiation-safety interlock device 122 configured to communicate with the optical detector 117 and the x-ray source 108, as indicated in FIG. 1 by dotted lines therebetween. The radiation-safety interlock device 122 may also be communicatively connected to the processor 116 (indicated in FIG. 1 by a dashed line therebetween).

During operation of the x-ray system 100, in some embodiments the optical system 114 is configured to detect, using the optical detector 117, a presence of an object at least one of in or covering the opening 105 defined by the table 102. The radiation-safety interlock device 122 allows the x-ray source 108 to be engaged while the presence of the object is detected, and disengages the x-ray source 108 otherwise. The object may be the breast 106 of the patient 103, a phantom for calibration and quality control, or a protective radiation shielding lid designed to seal the opening 105, for example.

For example, if the patient 103 is not on the table 102, or is improperly positioned thereupon so that her breast 106 is not fully extending through the opening 105 into the active spatial region, then operation of the x-ray source 108 could allow stray or scatter radiation to escape through the opening 105 in violation of radiation safety protocols and cause a safety hazard to the patient 103 as well as any other persons in the vicinity of the x-ray system 100. Therefore, the radiation-safety interlock device 122 is configured to only permit operation of the x-ray source 108 when the opening 105 is covered or occluded.

In some embodiments, the radiation-safety interlock device 122 analyzes a signal provided from the optical detector 117, and processes the signal to determine whether the opening 105 is empty, closed, or otherwise occluded by the breast 106 or some other object. In some embodiments, the radiation-safety interlock device 122 is communicatively connected to the processor 116, and receives a determination therefrom regarding whether the opening 105 is empty, closed, or otherwise occluded. The radiation-safety interlock device 122 then provides control instructions to the x-ray source 108 to enable or disable the x-ray source 108 based on the determination from the processor 116.

In some embodiments, the optical system 114 is configured to communicate with the x-ray source 108 to disable x-ray exposure based on at least one of the alignment of the breast 106 or the position of the breast 106.

During maintenance of the x-ray system 100, such as calibration of the x-ray assembly 107, an x-ray phantom (not shown) may be used instead of a patient 103, or a shield may be used to cover the opening 105. In these cases, the radiation-safety interlock device 122 may be used to ensure proper seating of the phantom or the shield cover, to again ensure that personnel in the vicinity of the x-ray system 100 are not accidentally exposed to x-ray radiation during the maintenance operations.

In some embodiments, a gantry assembly 123 is mounted upon the base component 101 and positioned beneath the table 102. The gantry assembly 123 includes a gantry platform 125 and a motor assembly 150 that is operatively connected to the rotatable x-ray assembly 107. The motor assembly 150 exerts a torque upon the gantry platform 125 in order to rotate the gantry platform 125 relative to the base component 101 about the axis of rotation 113 around the breast 106 and the surrounding active spatial region. In some embodiments, the x-ray assembly 107, including the x-ray source 108 and the x-ray detector 112, is rigidly mounted to the gantry platform 125 so as to also rotate around the breast 106 during rotation of the gantry platform 125 by the motor assembly 150.

In operation, the x-ray system 100 is configured to irradiate the breast 106 with the x-ray beam 110 generated by the x-ray source 108. The incident x-ray beam 110 is differentially attenuated by the tissues of the breast 106 in a spatially-varying manner before incidence on the x-ray detector 112. As the x-ray assembly 107 rotates around the axis of rotation 113 (e.g., by being rigidly mounted to the gantry platform 125, which is subject to a rotational torque applied by the motor assembly 150), different views of the breast 106 are exposed to the x-ray source 108 causing variable patterns of spatial attenuation in the incident x-ray beam 110. The x-ray detector 112 records the variations in the incident x-ray beam 110 during the rotation of the x-ray assembly 107 around the breast 106 and provides corresponding signals to the processor 116 which performs image processing on the received signals to generate one or more x-ray images of the breast 106.

In some embodiments, the x-ray system 100 may have a static subsystem (e.g., stationary components of the x-ray system 100 including but not limited to the table 102) and a rotating subsystem (e.g., rotatable components of the x-ray system 100 including but not limited to the x-ray source 108 and the x-ray detector 112) that is rotated by the motor assembly 150.

In some embodiments, the motor assembly 150 is a linear motor assembly. The linear motor assembly 150 is an electromagnetic motor that includes components such as magnet rails and magnetic coils. In some embodiments, the linear motor assembly 150 may also include a position encoder (not shown in FIG. 1), e.g., an external high-precision encoder. The encoder may also provide signals to the processor 116 that may be used with the signals from the x-ray detector 112 in order to generate x-ray images of the breast 106. The linear motor assembly 150 may also include a bearing that is capable of providing stiffness for the rotating subsystem based on the forces and moment load applied to it.

In some embodiments, the x-ray system 100 also includes a rigid enclosure (not shown in FIG. 1) that substantially encloses the x-ray source 108, the x-ray detector 112, and the active spatial region. Accordingly, during operation of the x-ray system 100, the enclosure also substantially encloses the x-ray beam 110 generated by the x-ray source 108. In some embodiments, the enclosure is a radiation shield enclosure that attenuates x-rays from the x-ray source 108 sufficiently for persons to be in proximity to the x-ray system 100 without further shielding during operation, while still complying with radiation safety standards. The shield enclosure may also in some embodiments be rigidly mounted to the gantry platform 125, and thereby rotate around the breast 106 with the x-ray source 108 and the x-ray detector 112.

In the example of FIG. 1, the optical detector 117 is mounted to the gantry platform 125 and rotates therewith. In other embodiments, the optical detector 117 may be mounted to the rigid enclosure of the x-ray system 100.

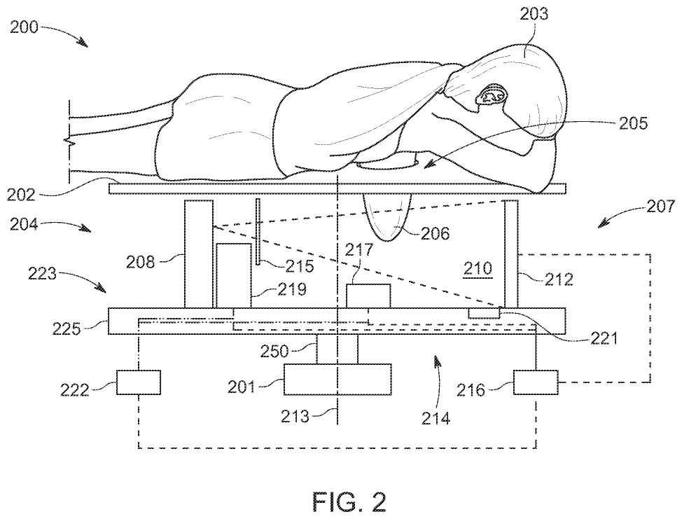

FIG. 2 shows another example of an x-ray system 200 for at least one of breast examinations and procedures, according to some embodiments of the current invention. The x-ray system 200 is similar to the embodiment of the x-ray system 100 discussed above with respect to FIG. 1, and the same or like reference numerals may be used to refer to equivalent or similar components. A detailed description of some of these components will be omitted, and the following discussion focuses on the differences between these embodiments. Any of the various features discussed with any one of the embodiments discussed herein may also apply to and be used with any other embodiments.

The x-ray system 200 includes a base component 201 and a table 202 configured to support a patient 203 in a prone position, the table being disposed proximate to the base component 201 with a space 204 reserved therebetween. The table 202 defines an opening 205 therein that is positioned for a breast 206 of the patient 203 to extend downwards therethrough at least partially into an active spatial region.

The x-ray system 200 also includes a rotatable x-ray assembly 207 disposed in the space reserved between the base component 201 and the table 202. In some embodiments, the base component 201 and the table 202 are configured to be fixed relative to each other, defining the space 104 therebetween to accommodate the rotatable x-ray assembly 207.

The rotatable x-ray assembly 207 includes an x-ray source 208 that generates an x-ray beam 210, and an x-ray detector 212 that is positioned to receive the x-ray beam 210. The x-ray assembly 207 is configured to rotate the x-ray source 208 and the x-ray detector 212 at least partially around the active spatial region about an axis of rotation 213.

In some embodiments, the x-ray system 200 may be configured to perform cone-beam computed tomography (CBCT). In some such embodiments, the x-ray source may be a CT x-ray tube, the x-ray detector 212 may be a flat panel detector, and the x-ray beam 210 generated by the x-ray source 208 may be a cone beam.

In some embodiments, the axis of rotation 213 does not intersect the breast 206. In other embodiments, the position of the opening 205 and the table 202 may be configured so that the axis of rotation 213 intersects at least part of the breast 206 as the breast 206 extends downwards into the active spatial region.

The x-ray source 208 is positioned to irradiate, with the x-ray beam 210, at least a portion of the active spatial region into which the breast 206 extends. The x-ray detector 212 is positioned to receive at least a portion of the incident x-ray beam 210 after passing through the active spatial region and at least a portion of the breast 206. The x-ray beam 210 may be collimated by a collimator 215 before irradiating at least a portion of the breast 206 and thereafter impinging on the x-ray detector 212. The x-ray detector 212 may also be communicatively connected to a processor 216 which receives signals from the x-ray detector 212 and processes the signals received therefrom, in order to, for example, generate x-ray images of the breast 206.

The x-ray system 200 includes an optical system 214 having a first optical detector 217 arranged in an optical path to the opening 205 of the table 202. The optical system 214 may also include the processor 216 in some embodiments. The optical path between the first optical detector 217 and the opening 205 of the table 202 may be parallel to the axis of rotation 213. In the example shown in FIG. 2, the first optical detector 217 is not aligned with the axis of rotation 213. However, in other embodiments, the first optical detector 217 may be aligned with or substantially coincide with the axis of rotation 213.

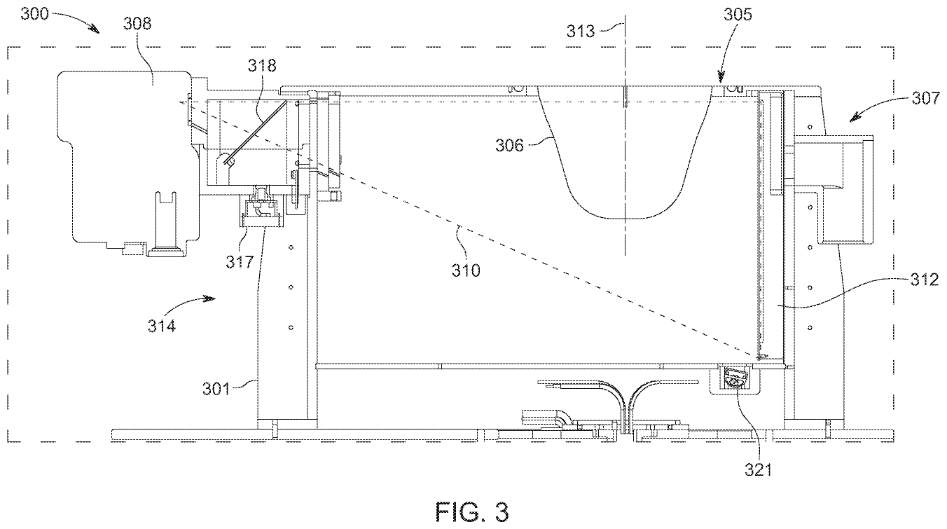

The optical system 214 of the x-ray system 200 also includes a second optical detector 219, arranged with an imaging plane (e.g., an optical field-of-view) that is optically conjugate with a radiation emission region of the x-ray source 208. In other words, the second optical detector 219 has a field of view that includes at least a majority of the active spatial region defined by the x-ray beam 210 during operation of the x-ray source 208. The second optical detector 219 may be positioned to the side, above, or below the aperture for the x-ray source 208, and use a mirror (see FIG. 3) to attain the same FOV as the x-ray source 208.

In some embodiments, the first optical detector 217, the second optical detector 219, or both are attached to the rotatable x-ray assembly 207 so as to rotate about the axis of rotation 213 in a substantially fixed relationship to the x-ray source 208 and the x-ray detector 212.

In some embodiments, the first optical detector 217, the second optical detector 219, or both are imaging optical detectors (e.g., optical cameras, an infrared cameras, etc.) that are each configured to provide an image of the breast 206, the opening 205, or both, within a respective imaging field of view. For example, the first optical detector 217, the second optical detector 219, or both may be communicatively connected to the processor 216 (as indicated in FIG. 2 by dashed lines therebetween), that receives and processes imaging data from the first optical detector 217, the second optical detector 219, or both, to generate optical images therefrom.

In some embodiments, the first optical detector 217, the second optical detector 219, or both are optical sensors that are each configured to provide a signal that varies based on how much light can be detected through the opening 205, depending on whether the opening 205 is empty, closed by a cover or shield, or at least partially occluded by the breast 206 of the patient 203 or by another object (e.g., a calibration phantom, etc.). The first optical detector 217, the second optical detector 219, or both may be communicatively connected to the processor 216 (as indicated in FIG. 2 by dashed lines therebetween), to receive and process the signal from the first optical detector 217, the second optical detector 219, or both.

During operation of the x-ray system 200, in some embodiments the optical system 214 is configured to detect, using either or both of the first optical detector 217 and the second optical detector 219, a presence of an object at least one of in or covering the opening 205 defined by the table 202.

In some embodiments, the optical system 214 also includes an illumination system 221 arranged to illuminate the breast 206, the x-ray detector 212, or both. The illumination system 221 provides enough illumination for either or both of the first optical detector 217 and the second optical detector 219 to optically detect the breast, even when the x-ray system 200 is scaled and a patient 203 is on the table 202.

In operation, in some embodiments the optical system 214 includes an image processor 222 that is configured to communicate (as indicated by the dotted lines in FIG. 2) with the first optical detector 217 and the second optical detector 219, to provide information about the breast 206 of the patient 203 when the breast 206 is extended downwards through the opening 205 defined by the table 202. This information about the breast 206 may include, but is not limited to, alignment information, volume information, shape information, motion information, and size information.

For example, the optical system 214 may be used to estimate breast motion before, during (between acquisitions), and after acquisition of x-ray data using the x-ray assembly 207. Data or images generated by the image processor 222 of the optical system 214 may be used to compensate for motion in the x-ray 3D reconstruction, which may reduce or eliminate the need for the patient 203 to hold their breath during the x-ray scanning procedure. The breast movement detection could be used to warn the operator of the x-ray system 200 during or after the x-ray procedure, that the images might not be of good quality (due to motion, positioning error, etc.) and therefore not worth performing or continuing the 3D reconstruction, and suggesting to re-do the x-ray procedure instead.

As another example, the optical system 214 may be used to determine the shape of the breast 206. The shape of the breast 206 can be reconstructed by scanning the breast 206 during a full rotation of the gantry platform 225 and also verifying that the breast 206 is centered within the required tolerances (e.g., ±1 cm of the axis) in the field of view prior to performing the x-ray imaging procedure (e.g., running a CT scan). The optical 3D reconstruction does not necessarily need to be as precise as the x-ray imaging 3D reconstruction, since the optical acquisition can serve as a verification method prior to the x-ray imaging procedure. The number of images may be limited by the specifications (e.g., frames-per-second or FPS) of the first optical detector 217, the second optical detector 219, or both.

The 3D reconstruction shape can also be used to verify the size and volume of the breast 206, and verify or inform the x-ray technique. The x-ray technique includes, but is not limited to, x-ray acquisition parameters such as x-ray intensity (e.g., the x-ray voltage, the x-ray current, etc.), and x-ray duration (e.g., the exposure time, also referred to as the firing time). In some embodiments, the size and volume estimation may be used to eliminate the need of x-ray scout images.

As another example, the optical system 214 may be used to determine and verify the alignment of the breast 206, during initial setup of the patient 203 on the table 202, as well as subsequently during the x-ray imaging procedure. The optical system 214 may be used to verify the position of the breast 206 in the active spatial region, e.g., by using the first optical detector 217, the second optical detector 219, or both. The optical system 214 may also be used to verify the alignment of the breast 206 relative to the axis of rotation 213, e.g., by using the first optical detector 217, the second optical detector 219, or both. By providing a view of the patient 203 in position, the optical system 214 may reduce the diagnostic procedure's duration and thereby increase patient throughput.

In some embodiments, the optical system 214 is configured to communicate with the x-ray source 208 to disable x-ray exposure, based on at least one of the alignment of the breast 206 or the position of the breast 206.

As another example, the optical system 214 may be used to verify the angular position of the gantry platform 225 (e.g., as a non-limiting example, to within ±0.02 degrees or better). Errors in angular position may thereby be reduced, improving the data reconstruction for the x-ray procedure.

In the example shown in FIG. 2, the image processor 222 is a separate processor from the processor 216, and is communicatively coupled thereto, as indicated by a dashed line therebetween. In other various embodiments, the image processor 222 may be a component of the processor 216, the processor 216 may be a component of the image processor 222, or the image processor 222 and the processor 216 may be sub-processors of another processing unit.

In some embodiments, a gantry assembly 223 is mounted upon the base component 201 and positioned beneath the table 202. The gantry assembly 223 includes a gantry platform 225 and a motor assembly 250 that is operatively connected to the rotatable x-ray assembly 207. The motor assembly 250 exerts a torque upon the gantry platform 225 in order to rotate the gantry platform 225 relative to the base component 201 about the axis of rotation 213, around the breast 206 and the surrounding active spatial region. In some embodiments, the x-ray assembly 207, including the x-ray source 208 and the x-ray detector 212, is rigidly mounted to the gantry platform 225 so as to also rotate around the breast 206 during rotation of the gantry platform 225 by the motor assembly 250.

In operation, the x-ray system 200 is configured to irradiate the breast 206 with the x-ray beam 210 generated by the x-ray source 208. The incident x-ray beam 210 is differentially attenuated by the tissues of the breast 206 in a spatially-varying manner, before incidence on the detector 212. As the x-ray assembly 207 rotates around the axis of rotation 213 (e.g., by being rigidly mounted to the gantry platform 225, which is subject to a rotational torque applied by the motor assembly 250), different views of the breast 206 are exposed to the x-ray source 208, causing variable patterns of spatial attenuation in the incident x-ray beam 210. The detector 212 records the variations in the incident x-ray beam 210 during the rotation of the x-ray assembly 207 around the breast 206, and provides corresponding signals to the processor 216, which performs image processing on the received signals to generate one or more x-ray images of the breast 206.

In some embodiments, the x-ray system 200 may have a static subsystem (e.g., stationary components of the x-ray system 200 including but not limited to the table 202) and a rotating subsystem (e.g., rotatable components of the x-ray system 200 including but not limited to the x-ray source 208 and the x-ray detector 212) that is rotated by the motor assembly 250.

In some embodiments, the motor assembly 250 is a linear motor assembly, that is an electromagnetic motor that includes components such as magnet rails and magnetic coils. In some embodiments, the motor assembly 250 may also include a position encoder (not shown in FIG. 2), e.g., an external high-precision encoder. The encoder may also provide signals to the processor 216 that may be used with the signals from the x-ray detector 212 in order to generate x-ray images of the breast 206. The motor assembly 250 may also include a bearing that is capable of providing stiffness for the rotating subsystem, based on the forces and moment load applied to it.

In some embodiments, the x-ray system 200 also includes a rigid enclosure (not shown in FIG. 2) that substantially encloses the x-ray source 208, the x-ray detector 212, and the active spatial region. Accordingly, during operation of the x-ray system 200, the enclosure also substantially encloses the x-ray beam 210 generated by the x-ray source 208. In some embodiments, the enclosure is a radiation shield enclosure that attenuates x-rays from the x-ray source 208 sufficiently for persons to be in proximity to the x-ray system 200 without further shielding during operation, while still complying with radiation safety standards. The shield enclosure may also in some embodiments be rigidly mounted to the gantry platform 225, and thereby rotate around the breast 206 with the x-ray source 208 and the x-ray detector 212.

In the example of FIG. 2, the first optical detector 217, the second optical detector 219, or both are mounted to the gantry platform 225. In other embodiments, the first optical detector 217, the second optical detector 219, or both may be mounted to the rigid enclosure of the x-ray system 100.

Additional embodiments of optical systems for an x-ray system are now described, and wherever possible, like reference numerals have been used to refer to equivalent or similar components. Any of the various features discussed with any one of the embodiments discussed herein may also apply to and be used with any other embodiments.

FIG. 3 shows another example of an x-ray system 300 for breast examinations and procedures, according to some embodiments of the current invention. In this example, the x-ray system 300 is enclosed by a radiation shield enclosure 301 having an opening 305 for the patient's breast 306 to extend downwards into the active spatial region. The patient table and the patient (excepting the breast 306) are omitted from FIG. 3 for clarity. Though not shown in FIG. 3, the opening in the patient table aligns with the opening 305 in the radiation shield enclosure 301 to accommodate the breast 306.

The x-ray system 300 includes a rotatable x-ray assembly 307, which includes an x-ray tube 308 that generates an x-ray beam 310, and a flat-panel detector 312. The radiation shield enclosure 301 fully encloses the active spatial region, the x-ray tube 308, and the flat-panel detector 312. The opening 305 in the radiation shield enclosure 301 is aligned in this example with an axis of rotation 313 for the rotatable x-ray assembly 307.

The x-ray system 300 includes an optical system 314. The optical system 314 includes an optical camera 317 that is mounted to the radiation shield enclosure 301, and a mirror 318 that is utilized by the optical camera 317 to achieve an optical field-of-view (FOV) that is conjugate with the x-ray beam 210. The mirror 318 may be, for example, an acrylic mirror of 1.5 mm thickness so as to minimize attenuation of the x-ray beam 310. The optical FOV includes the breast 306 and the flat-panel detector 312.

In addition, the optical system 314 includes a light-emitting diode (LED) strip 321 that is mounted to the radiation shield enclosure 301, to provide sufficient illumination of the breast 306 for the optical camera 317 to acquire optical images thereof. In some embodiments, the LED strip 321 may be tilted towards the flat-panel detector 312 to illuminate the background behind the breast 306, and thereby provide a greater contrast for breast image contour detection and reconstruction. In some embodiments, an x-ray-transparent film of a specific optical characteristic (e.g., a specific color or a reflection) may be used to enhance optical contrast, the optical characteristic depending in part upon the characteristics of the optical camera 317.

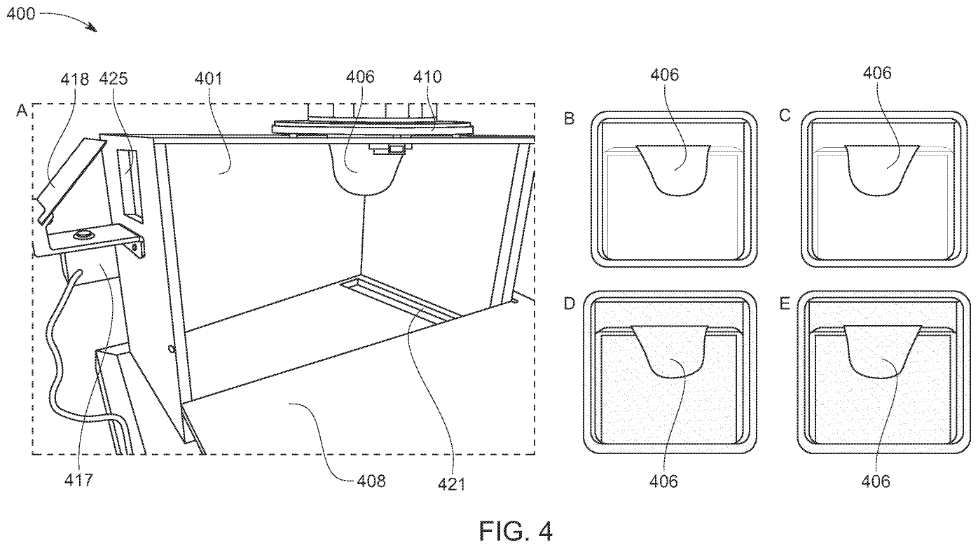

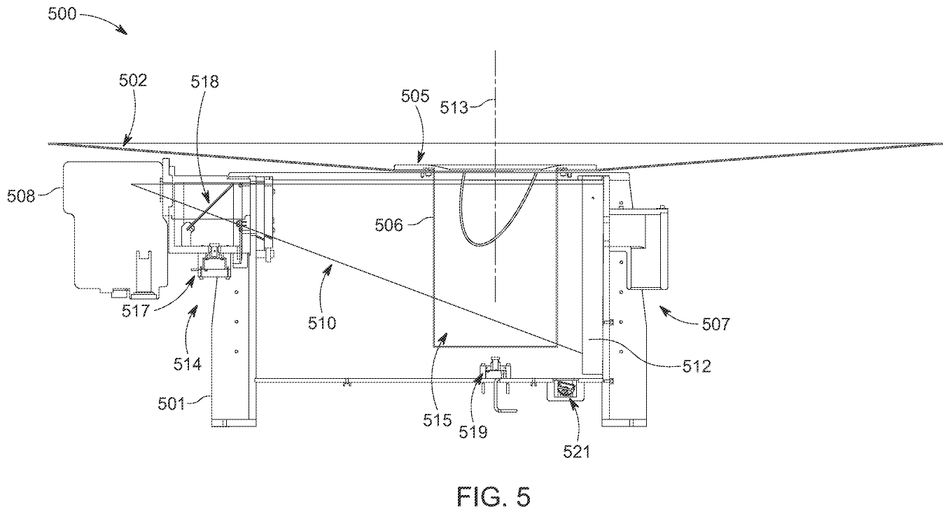

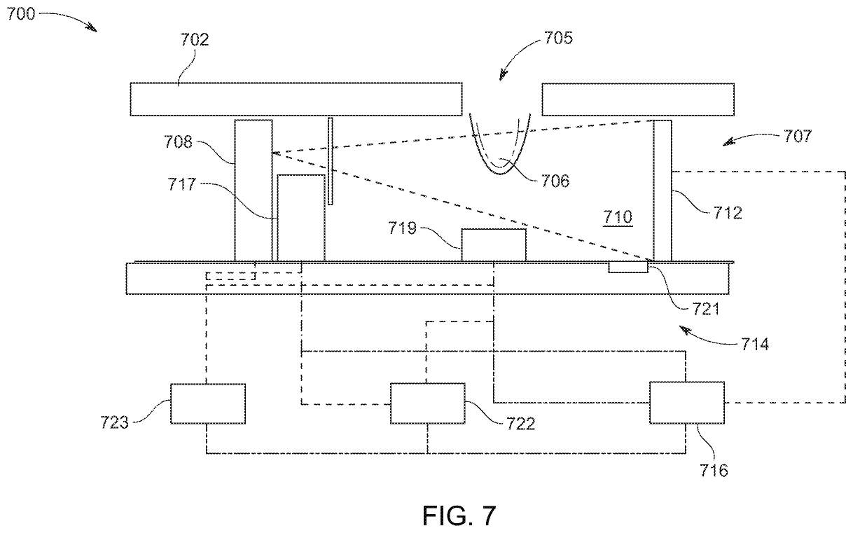

FIG. 4 shows an example of an optical system 400, according to some embodiments of the current invention. The optical system 400 may be used, as non-limiting examples, for some or all of the optical system 114 in x-ray system 100 (FIG. 1), the optical system 214 in x-ray system 200 (FIG. 2), the optical system 314 in x-ray system 300 (FIG. 3), the optical system 514 in x-ray system 500 (FIG. 5), the optical system 714 in x-ray system 700 (FIG. 7), or components thereof. In this example, panel A shows the optical system 400, enclosed by a scaled, light-proof enclosure 401 having an opening for a breast phantom 406 to extend downwards into the active spatial region. To illustrate the interior of the enclosure 401, one side wall 408 has been temporarily removed in this view. To simulate the rotation of a gantry, the breast phantom 406 is on a rotatable base 410. An optical camera 417 is positioned at one end of the enclosure 401, with an acrylic mirror 418 positioned outside an aperture 425 of the enclosure 401, to provide a view into the active spatial region. A gap 421 in the enclosure is also provided for an illumination system (not shown), to provide sufficient illumination when the enclosure 401 is fully sealed.

Panel B shows a first optical image of the breast phantom 406 acquired by the optical camera 417, while the rotatable base 410 is at a first angular orientation. Panel C shows a second optical image of the breast phantom 406 acquired by the optical camera 417, while the rotatable base 410 is at a second angular orientation. Panels D and E show the first and second optical images of the breast phantom 406, respectively, after undergoing image processing to detect edges and outlines of the breast phantom 406, and thereby facilitate position, volume, and shape estimation.

FIG. 5 shows another example of an x-ray system 500 for breast examinations and procedures, according to some embodiments of the current invention. In this example, the x-ray system 500 is enclosed by a radiation shield enclosure 501, and a patient table 502 is shown having an opening 505 for the patient's breast 506 to extend downwards into the active spatial region. Only the breast 506 is shown, with the rest of the patient omitted from FIG. 5 for clarity. The opening 505 in the patient table 502 aligns with an opening in the radiation shield enclosure 501 to accommodate the breast 506.

The x-ray system 500 includes a rotatable x-ray assembly 507, which includes an x-ray tube 508 that generates an x-ray beam 510, and a flat-panel detector 512. The radiation shield enclosure 501 fully encloses the active spatial region, the x-ray tube 508, and the flat-panel detector 512. The opening 505 is here aligned with the axis of rotation 513. The x-ray system 500 also includes an optical system 514, which is described below. In this example, a clear protective cup 515 is also positioned to fully enclose the breast 506 within the active spatial region. The clear protective cup 515 may be made of a material that does not substantially attenuate x-rays and optical light, such as acrylic.

The optical system 514 includes a first, “beam-view” optical camera 517 that is mounted to the side of the radiation shield enclosure 501, and a mirror 518 that is utilized by the beam-view optical camera 517 to achieve an optical field of view (FOV) that is conjugate with the x-ray beam 510. The mirror 518 may be, for example, an acrylic mirror of 1.5 mm thickness so as not to substantially attenuate the x-ray beam 510. The optical FOV includes the breast 506 and the flat-panel detector 512.

The optical system 514 also includes a second, “bottom-view” optical camera 519 that is mounted to the bottom of the radiation shield enclosure 501, such that the optical FOV of the bottom-view optical camera 519 is aligned with the axis of rotation 513. In addition, the optical system 514 also includes a light-emitting diode (LED) strip 521 that is mounted to the radiation shield enclosure 501, to provide sufficient illumination of the breast 506 for the beam-view optical camera 517 and the bottom-view optical camera 519 to acquire optical images thereof. In some embodiments, the LED strip 521 may be tilted towards the flat-panel detector 512 to illuminate the background behind the breast 506 to provide a greater contrast for breast image contour detection and reconstruction. In some embodiments, an x-ray-transparent film of a specific optical characteristic (e.g., a specific color or a reflection) may be used to enhance the optical contrast, the optical characteristic depending in part upon the characteristics of the beam-view optical camera 517, the bottom-view optical camera 519, or both.

In some embodiments, the bottom-view optical camera 519 may be used to verify that the breast 506 is centered within the opening 505. The bottom-view optical camera 519 could also be used to detect and visualize folds in the breast 506, and therefore assist the operator of the x-ray system 500 to correct the positioning of the breast 506 in the opening 505. In such a positioning procedure, the operator may guide the patient to position themselves through verbal instruction, as a non-contact procedure, or the operator may manually adjust the patient's position. The combination of the beam-view optical camera 517 and the bottom-view optical camera 519 may improve the initial breast position setup before beginning the x-ray procedure.

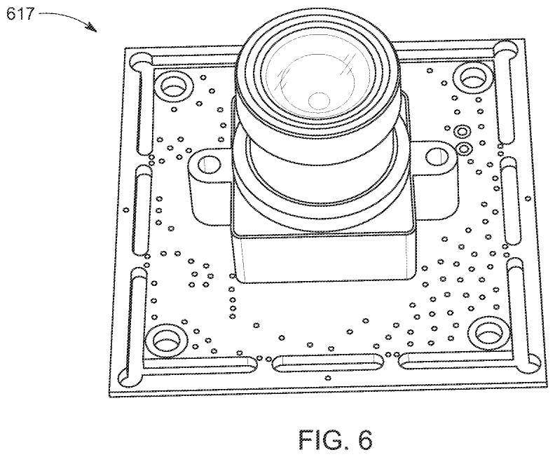

FIG. 6 shows an example of an imaging optical detector 617, according to some embodiments of the current invention. In this example, the imaging optical detector 617 is an optical camera. The imaging optical detector 617 may have the ability to acquire data at optical wavelengths as well as in low-light conditions, e.g., infrared wavelengths. The imaging optical detector 617 may be used, as non-limiting examples, for some or all of the optical detector 117 (FIG. 1), the first optical detector 217 (FIG. 2), the second optical detector 219 (FIG. 2), the optical camera 317 (FIG. 3), the optical camera 417 (FIG. 4), the beam-view optical camera 517 (FIG. 5), the bottom-view optical camera 519 (FIG. 5), the first optical detector 717 (FIG. 7), and the second optical detector 719 (FIG. 7).

FIG. 7 shows another example of an x-ray system 700 for breast examinations and procedures, according to some embodiments of the current invention. The x-ray system 700 includes a table 702 configured to support a patient in a prone position. The table 702 defines an opening therein that is positioned for a breast 706 of the patient to extend downwards therethrough at least partially into radiation emission region of an x-ray assembly 707 of the x-ray system 700.

The x-ray assembly 707 includes at least an x-ray source 708 that generates an x-ray beam 710, and an x-ray detector 712 that is positioned to receive the x-ray beam 710. For the sake of clarity, only the breast 706 is shown, and the patient is omitted, in FIG. 7.

In some embodiments, the x-ray system 700 may be configured to perform cone-beam computed tomography (CBCT). In some such embodiments, the x-ray source 708 may be a CT x-ray tube, the x-ray detector 712 may be a flat panel detector, and the x-ray beam 710 generated by the x-ray source 708 may be a cone beam. The x-ray system 700 also includes an optical system 714, which is described below.

The x-ray source 708 is positioned to irradiate, with the x-ray beam 710, at least a portion of the active spatial region into which the breast 706 extends. The x-ray detector 712 is positioned to receive at least a portion of the incident x-ray beam 710 after passing through the active spatial region and at least a portion of the breast 706. The x-ray detector 712 may also be communicatively connected to a processor 716 which receives signals from the x-ray detector 712 and processes the signals received therefrom, in order to, for example, generate x-ray images of the breast 706. The optical system 714 may include the processor 716 in some embodiments.

The optical system 714 includes a first optical detector 717 configured to be arranged with an imaging plane that is optically conjugate with a radiation emission region of an x-ray source 708 of said x-ray system 700. In other words, the first optical detector 717 has a field of view that includes at least a majority of the active spatial region defined by the x-ray beam 710 during operation of the x-ray source 708. The first optical detector 717 may be positioned to the side, above, or below the aperture for the x-ray source 708, and use a mirror (not shown) to attain the same FOV as the x-ray source 708.

The optical system 714 also includes a second optical detector 719 configured to be arranged in an optical path to the opening 705 defined by the table 702 of the x-ray system 700. The optical path between the second optical detector 719 and the opening 705 of the table 702 may in some embodiments substantially coincide with an axis of rotation of the x-ray assembly 707.

In some embodiments, the first optical detector 717, the second optical detector 719, or both are attached to the x-ray assembly 707 so as to rotate about the axis of rotation in a substantially fixed relationship to the x-ray source 708 and the x-ray detector 712.

The optical system 714 also includes an image processor 722 that is communicatively connected to the first optical detector 717 and the second optical detector 719, as indicated by dashed lines therebetween in FIG. 7. The image processor 722 is configured to communicate with the first optical detector 717 and the second optical detector 719 to provide information of the breast 706 of the patient when the breast 706 is extended downwards through the opening 705 defined by the table 702.

In some embodiments, the first optical detector 717, the second optical detector 719, or both are imaging optical detectors (e.g., optical cameras, an infrared cameras, etc.) that are each configured to provide an image of the breast 706, the opening 705, or both, within a respective imaging field of view. For example, the first optical detector 717, the second optical detector 719, or both may be communicatively connected to the processor 716 (as indicated in FIG. 7 by dotted lines therebetween), that receives and processes imaging data from the first optical detector 717, the second optical detector 719, or both, to generate optical images therefrom.

In some embodiments, the first optical detector 717, the second optical detector 719, or both are optical sensors that are each configured to provide a signal that varies based on how much light can be detected through the opening 705, depending on whether the opening 705 is empty, closed by a cover or shield, or at least partially occluded by the breast 706 of the patient or by another object (e.g., a calibration phantom, etc.). The first optical detector 717, the second optical detector 719, or both may be communicatively connected to the processor 716 (as indicated in FIG. 2 by dotted lines therebetween), to receive and process the signal from the first optical detector 717, the second optical detector 719, or both.

During operation of the x-ray system 700, in some embodiments the optical system 714 is configured to detect, using either or both of the first optical detector 717 and the second optical detector 719, a presence of an object at least one of in or covering the opening 705 defined by the table 702.

In some embodiments, the optical system 714 also includes an illumination system 721 arranged to illuminate the breast 706, the x-ray detector 712, or both. The illumination system 721 provides enough illumination for either or both of the first optical detector 717 and the second optical detector 719 to optically detect the breast, even when the x-ray system 700 is sealed and a patient is on the table 702.

In operation, in some embodiments the image processor 722 is configured to communicate (as indicated by the dashed lines in FIG. 7) with the first optical detector 717 and the second optical detector 719, to provide information about the breast 706 of the patient when the breast 706 is extended downwards through the opening 705 defined by the table 702. This information about the breast 706 may include, but is not limited to, alignment information, volume information, shape information, motion information, and size information.

In some embodiments, the optical system 714 is configured to communicate with the x-ray source 708 to disable x-ray exposure, based on at least one of the alignment of the breast 706 or the position of the breast 706.

For example, the optical system 714 may be used to estimate breast motion before, during (between acquisitions), and after acquisition of x-ray data using the x-ray assembly 707. Data or images generated by the image processor 722 of the optical system may be used to compensate for motion in the x-ray 3D reconstruction, which may reduce or eliminate the need for the patient to hold their breath during the x-ray scanning procedure. The breast movement detection could be used to warn the operator of the x-ray system 700 during or after the x-ray procedure, that the images might not be of good quality (due to motion, positioning error, etc.) and therefore not worth performing or continuing the 3D reconstruction, and suggesting to re-do the x-ray procedure instead.

As another example, the optical system 714 may be used to determine the shape of the breast 706. The shape of the breast 706 can be reconstructed by scanning the breast 706 during a full rotation of the x-ray assembly 707 and also verifying that the breast 706 is centered within the required tolerances (e.g., ±1 cm of the axis) in the field of view prior to performing the x-ray imaging procedure (e.g., running a CT scan). The optical 3D reconstruction does not need to be as precise as the x-ray imaging 3D reconstruction since the optical acquisition serves as a verification method prior to the x-ray imaging procedure. The number of images will be limited by the specifications (e.g., frames-per-second or FPS) of the first optical detector 717, the second optical detector 719, or both.

The 3D reconstruction shape can also be used to verify the size and volume of the breast 706, and verify or inform the x-ray technique. The x-ray technique includes, but is not limited to, x-ray acquisition parameters such as x-ray intensity (e.g., the x-ray voltage, the x-ray current, etc.), and x-ray duration (e.g., the exposure time, also referred to as the firing time). In some embodiments, the size and volume estimation may be used to eliminate the need of x-ray scout images.

As another example, the optical system 714 may be used to determine and verify the alignment of the breast 706, during initial setup of the patient on the table 702, as well as subsequently during the x-ray imaging procedure. The optical system may be used to verify the position of the breast 706 in the active spatial region, e.g., by using the first optical detector 717, the second optical detector 719, or both. The optical system 714 may also be used to verify the alignment of the breast 706 relative to the axis of rotation of the x-ray assembly 707. e.g., by using the first optical detector 717, the second optical detector 719, or both. By providing a view of the patient in position, the optical system 714 may reduce the diagnostic procedure's duration and thereby increase patient throughput.

As another example, the optical system 714 may be used to verify the angular position of the gantry platform 225 (e.g., within ±2 degrees). Errors in angular position may thereby be reduced, improving the data reconstruction for the x-ray procedure.

In the example shown in FIG. 7, the image processor 722 is a separate processor from the processor 716. In other various embodiments, the image processor 722 may be a component of the processor 716, the processor 716 may be a component of the image processor 722, or the image processor 722 and the processor 716 may be sub-processors.

In some embodiments, the optical system 714 may also include a radiation-safety interlock device 723 configured to communicate with at least one of the first optical detector 717 and the second optical detector 719, and the x-ray source 108, as indicated in FIG. 7 by dashed lines therebetween. The radiation-safety interlock device 723 may also be communicatively connected to at least one of the processor 716 and the image processor 722, as indicated in FIG. 7 by dotted lines therebetween.

During operation of the x-ray system 700, in some embodiments the optical system 714 is configured to detect, using at least one of the first optical detector 717 and the second optical detector 719, a presence of an object at least one of in or covering the opening 705 defined by the table 702. The radiation-safety interlock device 723 allows the x-ray source 708 to be engaged while the presence of the object is detected, and disengages the x-ray source 708 otherwise. The object may be the breast 706 of the patient, a phantom for calibration and quality control, or a protective radiation shielding lid designed to seal the opening 705.

For example, if the patient is not on the table 702, or is improperly positioned thereupon so that their breast 706 is not fully extending through the opening 705 into the active spatial region, then operation of the x-ray source 708 may allow stray or scatter radiation to escape through the opening 705 in violation of radiation safety protocols, and cause a safety hazard to the patient as well as any other persons in the vicinity of the x-ray system 700. Therefore, the radiation-safety interlock device 723 is configured to only permit operation of the x-ray source 708 when the opening 705 is covered or occluded.

In some embodiments, the radiation-safety interlock device 723 analyzes a signal provided from at least one of the first optical detector 717 and the second optical detector 719, and processes the signal to determine whether the opening 705 is empty, closed, or otherwise occluded by the breast 706 or some other object. In some embodiments, the radiation-safety interlock device 723 is communicatively connected to the processor 716, and receives a determination therefrom regarding whether the opening 705 is empty, closed, or otherwise occluded. The radiation-safety interlock device 723 then provides control instructions to the x-ray source 708 to enable or disable the x-ray source 708, based on the determination from the processor 716.

During maintenance of the x-ray system 700, such as calibration of the x-ray assembly 707, an x-ray phantom (not shown) may be used instead of a patient, or a shield may be used to cover the opening 705. In these cases, the radiation-safety interlock device 723 may be used to ensure proper seating of the phantom or the shield cover, to again ensure that personnel in the vicinity of the x-ray system 700 are not accidentally exposed to x-ray radiation during the maintenance operations.

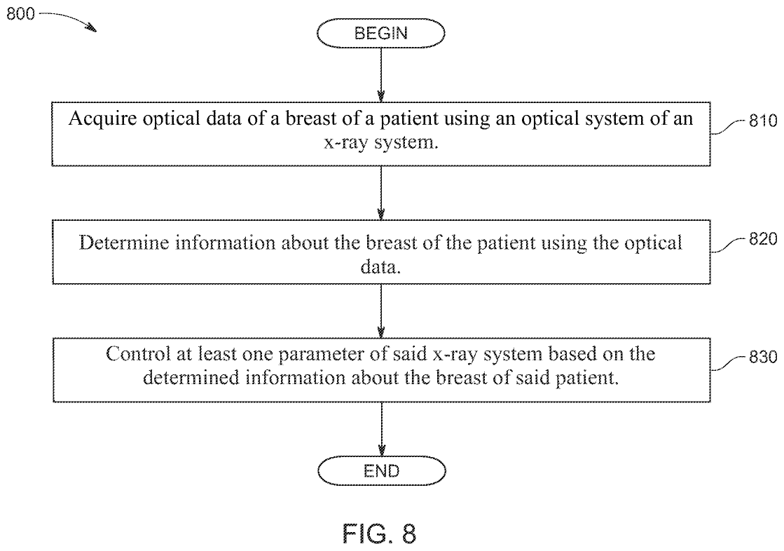

FIG. 8 illustrates a process 800 for performing at least one of breast examinations and procedures, according to some embodiments of the current invention. The process 800 may be performed, for example, by any of the processor 116 (FIG. 1), the processor 216 (FIG. 2), and the processor 716 (FIG. 7).

The process begins at 800 by providing an x-ray system that includes an optical system. The x-ray system may be, for example, any of x-ray system 100, x-ray system 200, x-ray system 300, x-ray system 500, and x-ray system 700. The optical system may be, for example, any of optical system 400 and optical system 714.

The process continues at 810 by acquiring optical data of a breast of a patient using the optical system of the x-ray system. The optical data is acquired, for example, while the breast extends downwards through an opening defined by a table of the x-ray system, the table being configured to support the patient.

In some embodiments, the optical data may be acquired from one or more optical detectors of the optical system. For example, the optical detectors may be any one of the optical detector 117 (FIG. 1), the first optical detector 217 (FIG. 2), the second optical detector 219 (FIG. 2), the optical camera 317 (FIG. 3), the optical camera 417 (FIG. 4), the beam-view optical camera 517 (FIG. 5), the bottom-view optical camera 519 (FIG. 5), the first optical detector 717 (FIG. 7), and the second optical detector 719 (FIG. 7). Each of the one or more optical detectors may be arranged in an optical path to the opening of the table, or arranged with an imaging plane (e.g., an optical field-of-view) that is optically conjugate with a radiation emission region of an x-ray source of the x-ray system.

The process continues at 820 by determining information about the breast of the patient using the optical data. For example, in some embodiments, the information about the breast of the patient may include at least one of a size of the breast, a shape of the breast, and a volume of the breast.

The process continues at 830 by controlling at least one parameter of the x-ray system, based on the determined information about the breast of the patient. The parameter of the x-ray system may be controlled during the at least one of breast examinations and procedures. For example, in some embodiments, controlling at least one parameter of the x-ray system includes controlling at least one of an x-ray intensity or an x-ray duration of an x-ray source of the x-ray system.

In some embodiments, the information about the breast of the patient includes at least one of an alignment of the breast and a position of the breast, and controlling at least one parameter of the x-ray system comprises disabling the x-ray system based on at least one of the alignment and the position.

The process 800 then ends.

The terms “light” and “optical” are intended to have broad meanings that can include both visible regions of the electromagnetic spectrum as well as other regions, such as, but not limited to, infrared and ultraviolet light and optical imaging, for example, of such light.

The terms “computer”, “server”, “processor”, and “memory” all refer to electronic or other technological devices. These terms exclude people or groups of people. As used in this specification, the terms “computer readable medium,” “computer readable media,” and “machine readable medium,” etc. are entirely restricted to tangible, physical objects that store information in a form that is readable by a computer. These terms exclude any wireless signals, wired download signals, and any other ephemeral signals.

The term “computer” is intended to have a broad meaning that may be used in computing devices such as, e.g., but not limited to, standalone or client or server devices. The computer may be, e.g., (but not limited to) a personal computer (PC) system running an operating system such as, e.g., (but not limited to) MICROSOFT® WINDOWS® available from MICROSOFT® Corporation of Redmond, Wash., U.S.A. or an Apple computer executing MAC® OS from Apple® of Cupertino, Calif., U.S.A. However, the invention is not limited to these platforms. Instead, the invention may be implemented on any appropriate computer system running any appropriate operating system. In one illustrative embodiment, the present invention may be implemented on a computer system operating as discussed herein. The computer system may include, e.g., but is not limited to, a main memory, random access memory (RAM), and a secondary memory, etc. Main memory, random access memory (RAM), and a secondary memory, etc., may be a computer-readable medium that may be configured to store instructions configured to implement one or more embodiments and may comprise a random-access memory (RAM) that may include RAM devices, such as Dynamic RAM (DRAM) devices, flash memory devices, Static RAM (SRAM) devices, etc.

The secondary memory may include, for example, (but not limited to) a hard disk drive and/or a removable storage drive, representing a floppy diskette drive, a magnetic tape drive, an optical disk drive, a read-only compact disk (CD-ROM), digital versatile discs (DVDs), flash memory (e.g., SD cards, mini-SD cards, micro-SD cards, etc.), read-only and recordable Blu-Ray® discs, etc. The removable storage drive may, e.g., but is not limited to, read from and/or write to a removable storage unit in a well-known manner. The removable storage unit, also called a program storage device or a computer program product, may represent, e.g., but is not limited to, a floppy disk, magnetic tape, optical disk, compact disk, etc. which may be read from and written to the removable storage drive. As will be appreciated, the removable storage unit may include a computer usable storage medium having stored therein computer software and/or data.

In some embodiments, the secondary memory may include other similar devices for allowing computer programs or other instructions to be loaded into the computer system. Such devices may include, for example, a removable storage unit and an interface. Examples of such may include a program cartridge and cartridge interface (such as, e.g., but not limited to, those found in video game devices), a removable memory chip (such as, e.g., but not limited to, an erasable programmable read only memory (EPROM), or programmable read only memory (PROM) and associated socket, and other removable storage units and interfaces, which may allow software and data to be transferred from the removable storage unit to the computer system.

Some embodiments include electronic components, such as microprocessors, storage and memory that store computer program instructions in a machine-readable or computer-readable medium (alternatively referred to as computer-readable storage media, machine-readable media, or machine-readable storage media). The computer-readable media may store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

The computer may also include an input device that may include any mechanism or combination of mechanisms that may permit information to be input into the computer system from, e.g., a user. The input device may include logic configured to receive information for the computer system from, e.g., a user. Examples of the input device may include, e.g., but not limited to, a mouse, a track pad, a pen-based pointing device, or other pointing device such as a digitizer, a touch sensitive display device, and/or a keyboard or other data entry device (none of which are labeled). Other input devices may include, e.g., but not limited to, a biometric input device, a video source, an audio source, a microphone, a web cam, a video camera, and/or another camera. The input device may communicate with a processor either wired or wirelessly.

The computer may also include output devices which may include any mechanism or combination of mechanisms that may output information from a computer system. An output device may include logic configured to output information from the computer system. Embodiments of output device may include, e.g., but not limited to, display, and display interface, including displays, printers, speakers, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), etc. The computer may include input/output (I/O) devices such as, e.g., (but not limited to) communications interface, cable and communications path, etc. These devices may include, e.g., but are not limited to, a network interface card, and/or modems. The output device may communicate with processor either wired or wirelessly. A communications interface may allow software and data to be transferred between the computer system and external devices.

The term “data processor” is intended to have a broad meaning that includes one or more processors, such as, e.g., but not limited to, that are connected to a communication infrastructure (e.g., but not limited to, a communications bus, cross-over bar, interconnect, or network, etc.). The term data processor may include any type of processor, microprocessor and/or processing logic that may interpret and execute instructions, including application-specific integrated circuits (ASICs) and field-programmable gate arrays (FPGAs). The data processor may comprise a single device (e.g., for example, a single core) and/or a group of devices (e.g., multi-core). The data processor may include logic configured to execute computer-executable instructions configured to implement one or more embodiments. The instructions may reside in main memory or secondary memory. The data processor may also include multiple independent cores, such as a dual-core processor or a multi-core processor. The data processors may also include one or more graphics processing units (GPU) which may be in the form of a dedicated graphics card, an integrated graphics solution, and/or a hybrid graphics solution. Various illustrative software embodiments may be described in terms of this illustrative computer system. After reading this description, it will become apparent to a person skilled in the relevant art(s) how to implement the invention using other computer systems and/or architectures.

The term “data storage device” is intended to have a broad meaning that includes removable storage drive, a hard disk installed in hard disk drive, flash memories, removable discs, non-removable discs, etc. In addition, it should be noted that various electromagnetic radiation, such as wireless communication, electrical communication carried over an electrically conductive wire (e.g., but not limited to twisted pair, CAT5, etc.) or an optical medium (e.g., but not limited to, optical fiber) and the like may be encoded to carry computer-executable instructions and/or computer data that embodiments of the invention on e.g., a communication network. These computer program products may provide software to the computer system. It should be noted that a computer-readable medium that comprises computer-executable instructions for execution in a processor may be configured to store various embodiments of the present invention.

The term “network” is intended to include any communication network, including a local area network (“LAN”), a wide area network (“WAN”), an Intranet, or a network of networks, such as the Internet.

The term “software” is meant to include firmware residing in read-only memory or applications stored in magnetic storage, which can be read into memory for processing by a processor. Also, in some embodiments, multiple software inventions can be implemented as sub-parts of a larger program while remaining distinct software inventions. In some embodiments, multiple software inventions can also be implemented as separate programs. Finally, any combination of separate programs that together implement a software invention described here is within the scope of the invention. In some embodiments, the software programs, when installed to operate on one or more electronic systems, define one or more specific machine implementations that execute and perform the operations of the software programs.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described. Moreover, features described in connection with one embodiment may be used in conjunction with other embodiments, even if not explicitly stated above.

1. An x-ray system for at least one of breast examinations and procedures, comprising:
a base component;
a table configured to support a patient in a prone position and disposed proximate said base component with a space reserved therebetween, said table defining an opening that is positioned for a breast of said patient to extend downwards therethrough at least partially into an active spatial region;
a rotatable x-ray assembly disposed in the space reserved between said base component and said table, said rotatable x-ray assembly comprising an x-ray source and an x-ray detector and being configured to rotate said x-ray source and said x-ray detector at least partially around said active spatial region about an axis of rotation;
an optical system comprising an optical detector arranged in an optical path to said opening of said table; and
a radiation-safety interlock device configured to communicate with said optical detector and said x-ray source,
wherein said optical system is configured to detect whether said opening defined by said table is covered or occluded, and
wherein said radiation-safety interlock device is configured to only permit operation of the x-ray source when the opening is covered or occluded.

2. The x-ray system according to claim 1, wherein said optical detector of said optical system is arranged such that said optical path coincides substantially with said axis of rotation.

3. The x-ray system according to claim 2, wherein said optical detector of said optical system is an imaging optical detector arranged to image at least a portion of said breast of said patient when said breast is extended downwards through said opening defined by said table at least partially into said active spatial region.

4. The x-ray system according to claim 3, wherein said optical system further comprises an image processor configured to communicate with said optical detector to provide alignment information of said breast of said patient when said breast extends downwards through said opening defined by said table.

5. The x-ray system according to claim 4, wherein said optical detector of said optical system is attached to said rotatable x-ray assembly and rotates about said axis of rotation in a substantially fixed relationship to said x-ray source and said x-ray detector.

6. The x-ray system according to claim 3, wherein said optical detector is a first optical detector and said optical system further comprises a second optical detector,
wherein said second optical detector is a second optical imaging system arranged with an imaging plane that is optically conjugate with a radiation emission region of said x-ray source, and
wherein said second optical detector is attached to said rotatable x-ray assembly and rotates about said axis of rotation in a substantially fixed relationship to said x-ray source and said x-ray detector.

7. The x-ray system according to claim 6, wherein said optical system further comprises an image processor configured to communicate with said first optical detector and said second optical detector to provide at least one of a size of said breast, a shape of said breast, and a volume of said breast, when said breast is extended downwards through said opening defined by said table.

8. The x-ray system according to claim 7, wherein said optical system is further configured to communicate with said x-ray source to set at least one of an x-ray intensity and an x-ray duration based on said at least one of said size, said shape, and said volume.

9. The x-ray system according to claim 7, wherein the image processor is further configured to communicate with said second optical detector to provide at least one of an alignment of said breast and a position of said breast.

10. The x-ray system according to claim 9, wherein said optical system is further configured to communicate with said x-ray source to disable x-ray exposure based on at least one of said alignment and said position.

11. The x-ray system according to claim 1, wherein said optical detector is a first optical detector and said optical system further comprises a second optical detector,
wherein said second optical detector is an optical imaging system arranged with an imaging plane that is optically conjugate with a radiation emission region of said x-ray source, and
wherein said second optical detector is attached to said rotatable x-ray assembly and rotates about said axis of rotation in a substantially fixed relationship to said x-ray source and said x-ray detector.

12. The x-ray system according to claim 11, wherein said optical system further comprises an image processor configured to communicate with said second optical detector to provide at least one of a size of said breast, a shape of said breast, and a volume of said breast, when said breast is extended downwards through said opening defined by said table.

13. The x-ray system according to claim 12, wherein the image processor is further configured to communicate with said second optical detector to provide at least one of alignment information, shape information, and motion information of said breast.

14. An x-ray system for at least one of breast examinations and procedures, comprising:
a base component;
a table configured to support a patient in a prone position and disposed proximate said base component with a space reserved therebetween, said table defining an opening that is positioned for a breast of said patient to extend downwards therethrough at least partially into an active spatial region;
a rotatable x-ray assembly disposed in the space reserved between said base component and said table, said rotatable x-ray assembly comprising an x-ray source and an x-ray detector and being configured to rotate said x-ray source and said x-ray detector at least partially around said active spatial region about an axis of rotation; and
an optical system comprising a first imaging optical detector arranged with an imaging plane that is optically conjugate with a radiation emission region of said x-ray source and a second imaging optical detector arranged in an optical path to said opening of said table,
wherein said optical system further comprises an image processor configured to communicate with said first imaging optical detector and said second imaging optical detector to provide information of said breast of said patient when said breast is extended downwards through said opening defined by said table.

15. The x-ray system according to claim 14, wherein said information of said breast comprises at least one of alignment information, volume information, shape information, motion information, or size information.

16. The x-ray system according to claim 15, wherein said optical system is further configured to communicate with said x-ray source to set at least one of an x-ray intensity, position, duration, or firing time based on said information of said breast.

17. The x-ray system according to claim 15, wherein said first imaging optical detector and said second imaging optical detector are each attached to said rotatable x-ray assembly so as to rotate about said axis of rotation in a substantially fixed relationship to said x-ray source and said x-ray detector.

18. An optical system configured for an x-ray system, comprising:
a first imaging optical detector configured to be arranged with an imaging plane that is optically conjugate with a radiation emission region of an x-ray source of said x-ray system;
a second imaging optical detector configured to be arranged in an optical path to an opening defined by a table of said x-ray system, said table being configured to support a patient in a prone position, said opening being positioned for a breast of said patient to extend downwards therethrough at least partially into said radiation emission region; and
an image processor configured to communicate with said first imaging optical detector and said second imaging optical detector to provide information of said breast of said patient when said breast is extended downwards through said opening defined by said table.

19. The optical system according to claim 18, wherein said information of said breast comprises at least one of alignment information, volume information, shape information, motion information, and size information.

20. The optical system according to claim 19, wherein said optical system is further configured to communicate with said x-ray source to set at least one of an x-ray intensity, position, duration, or firing time based on said information of said breast.

21. The optical system according to claim 19, wherein said first imaging optical detector and said second imaging optical detector are each configured to be attached to a rotatable x-ray assembly disposed beneath said table,
wherein said rotatable x-ray assembly comprises said x-ray source and an x-ray detector, and
wherein said first imaging optical detector and said second imaging optical detector are each attached to said rotatable x-ray assembly so as to rotate about said axis of rotation in a substantially fixed relationship to said x-ray source and said x-ray detector.

22. The optical system according to claim 21, further comprising an optical illumination system arranged to illuminate at least one of said breast of said patient and a panel of said x-ray detector.

23. (canceled)

24. (canceled)

25. (canceled)

26. The x-ray system according to claim 1, wherein said optical system is configured to detect whether said opening defined by said table is covered or occluded based on detecting an amount of light passing through said opening defined by said table.
